(19) United States
(12) Plant Patent Application Publication
(10) Pub. No.: US 2005/0039238 P1
(43) Pub. Date: Feb. 17, 2005

(54) Osteospermum plant named 'Sunny Philip'

(50) OSTEOSPERMUM ECKLONIS; OSTEOSPERMUM

(75) Inventor: Bjarne Nyholm Larsen, Odense N (DK)

Correspondence Address: FOLEY AND LARDNER;SUITE 500, 3000 K STREET NW, WASHINGTON, DC, 20007, US

(73) Assignee: SUNNY Osteospermum ApS

(21) Appl. No.: 10/873,295

(22) Filed: Jun. 23, 2004

(30) Foreign Application Priority Data
Jul. 7, 2003 (QZ) 2003/1238

(51) Int. Cl. A01H 5/00

(52) U.S. Cl. PLT/360

(57) ABSTRACT

A new distinct cultivar of Osteospermum plant named ‘Sunny Philip’, characterized by its large upright purple flower heads; spatulate ray florets with colors: from white RHS N155B to violet RHS 85A on top and blue-violet on the back RHS 86A to RHS 86C; compact and bushy plant form, mainly due to more upright stems; moderately vigorous growth habit, but less need for chemical growth retardation; and high number of large flowers per plant.

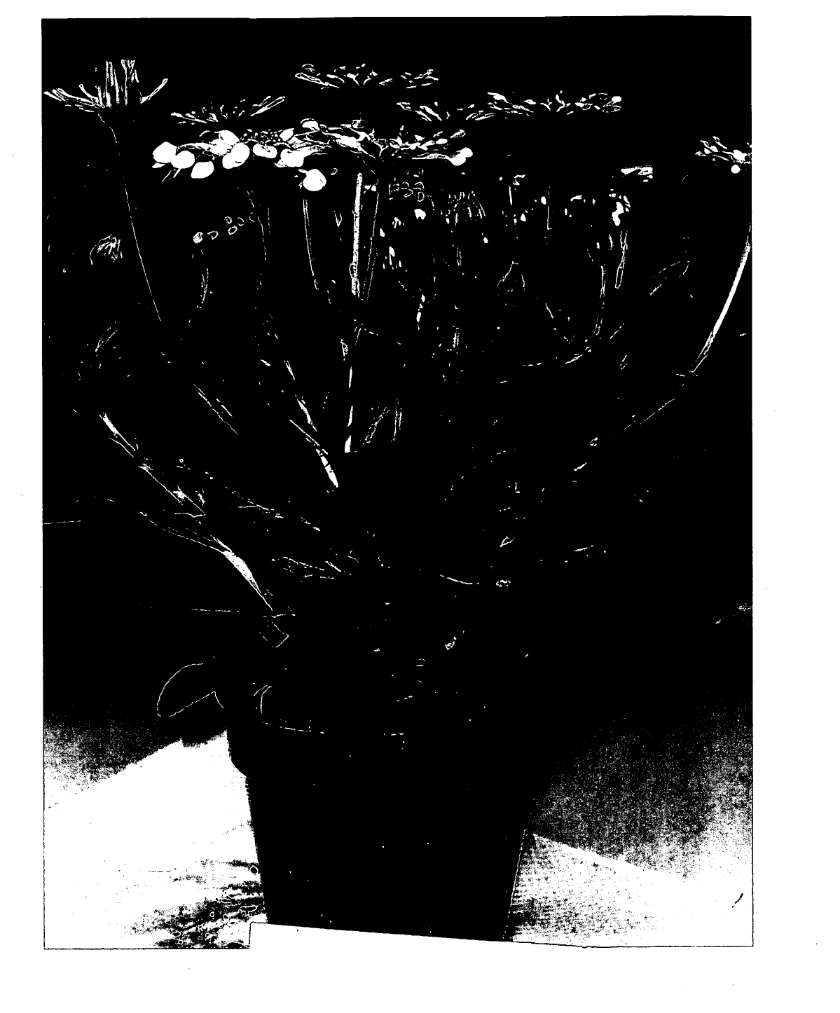

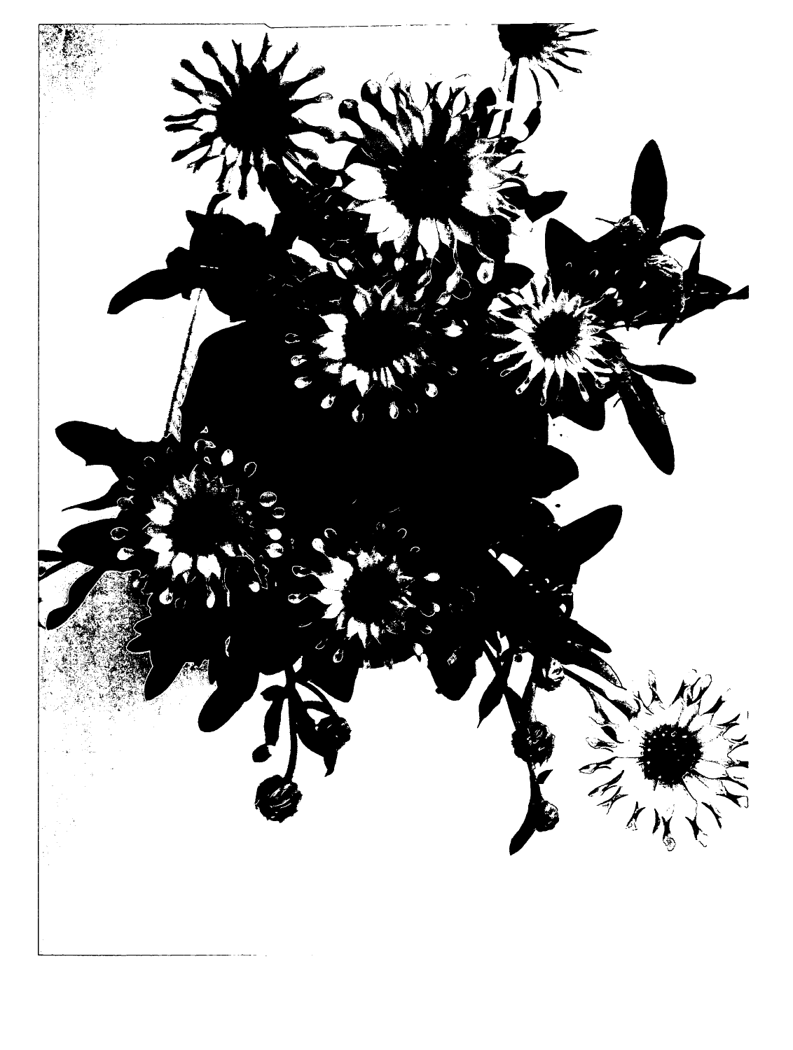

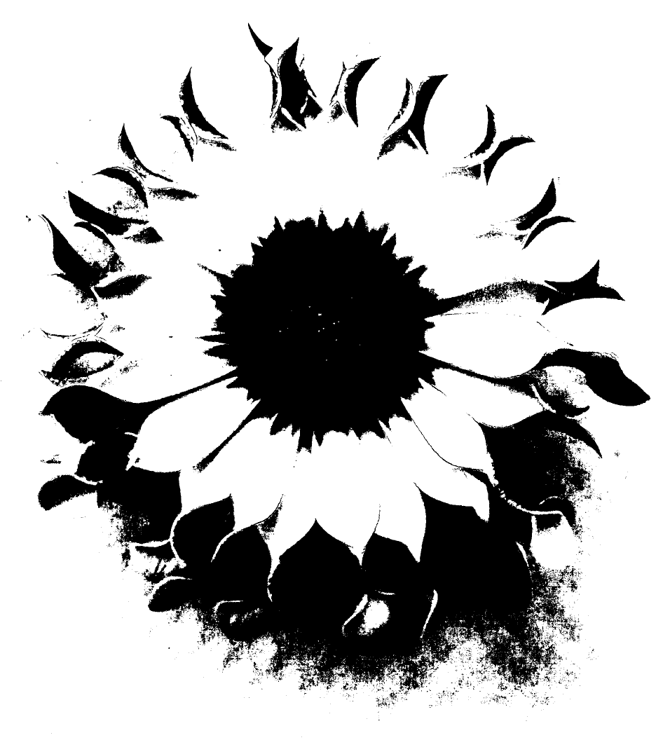

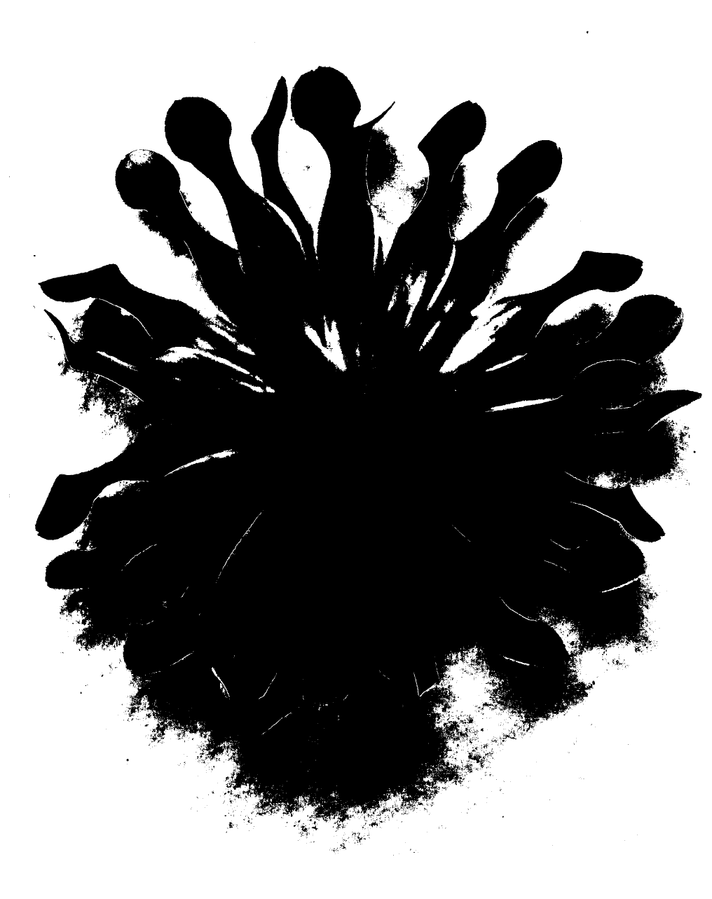

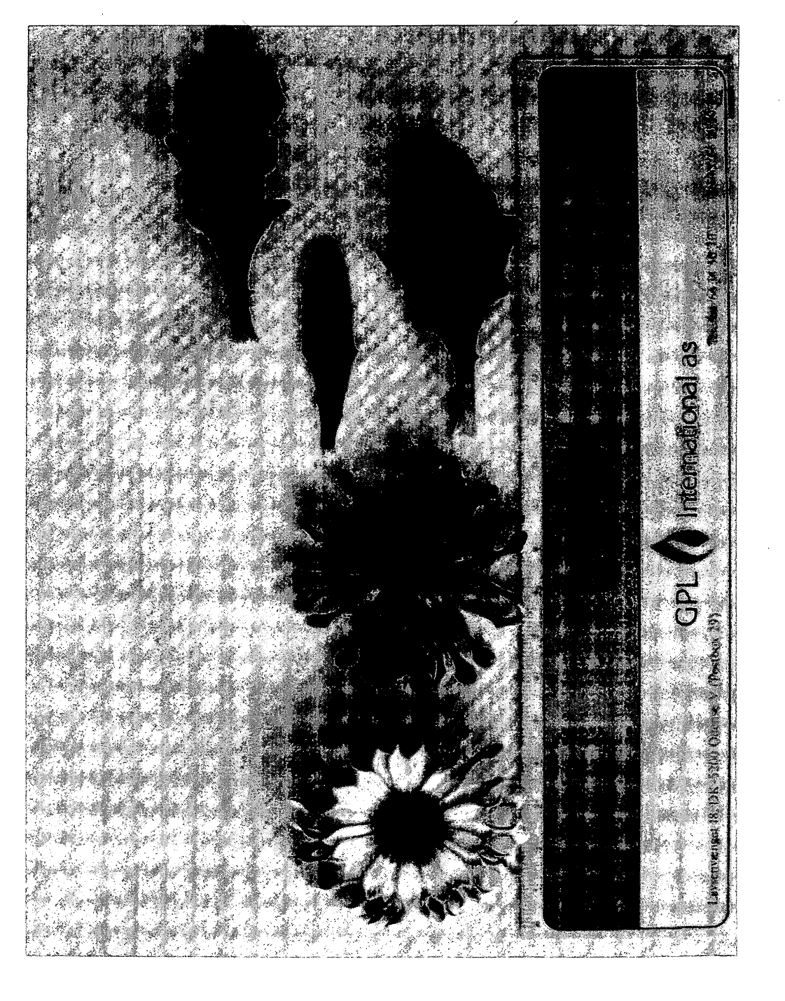

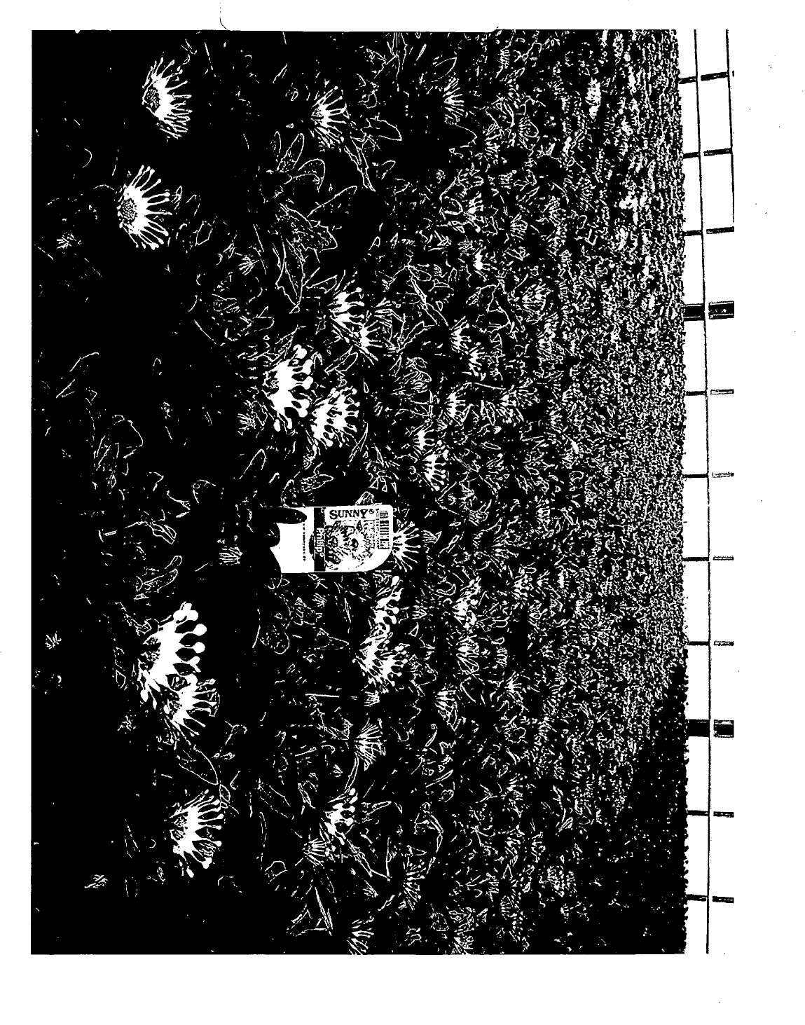

### GENUS AND SPECIES OF THE PLANT CLAIMED

Osteospermum eclonis (DC) T. Norl.

### VARIETY DENOMINATION

Sunny Philip

### BACKGROUND OF THE INVENTION

The present Invention relates to a new and distinct cultivar of Osteospermum plant, botanically known as Osteospermum eclonis (DC) T. Norl., commonly known as Cape Daisy and hereinafter referred to by the variety denomination ‘Sunny Philip’.

The new Osteospermum is a product of a planned breeding program conducted by the Inventor, Bjarne Larsen, in Stige, Denmark. The new Osteospermum originated from a controlled cross breeding made in May 2000 by the Inventor between between Osteospermum eclonis (DC) T. Norl. seedling designated ‘3.122.983’ and Osteospermum eclonis (DC) T. Norl. seedling designated ‘9913’. The Inventor selected the new Osteospermum cultivar from the progeny of the above crossing in 2003 on the basis of its flower color, size, and compact, freely branching habit. Plants of the new Osteospermum are upright, compact and have a unique color combination of upper and underside of the ray petals.

Asexual reproduction of the new cultivar by terminal cuttings taken and propagated at trial production batches in Stige, Denmark, has shown that the unique features of this new Osteospermum are stable and reproduced true to type in many successive generations.

### SUMMARY OF THE INVENTION

The following traits have been repeatedly observed and are determined to be the unique characteristics of ‘Sunny Philip’. These characteristics in combination distinguish ‘Sunny Philip’ as a new and distinct cultivar:

1. Large upright purple flower heads;

2. Spatulate ray florets with colors: from white RHS N155B to violet RHS 85A on top and blue-violet on the back RHS 86A to RHS 86C;

3. Compact and bushy plant form, mainly due to more upright stems;

4. Moderately vigorous growth habit, but less need for chemical growth retardation; and

5. High number of large flowers per plant.

Plants of the cultivar ‘Sunny Philip’ can be compared to plants of the Osteospermum eclonis (DC) T. Norl. Cultivar ‘Sunny Zara’ (unpatented). However, in side-by-side comparisons conducted by the Inventor in Stige Denmark, plants of the cultivar ‘Sunny Philip’ and the cultivar ‘Sunny Zara’ differ in the following characteristics:

1. Plants of the new Osteospermum have striking color combinations of white/violet ray flowers and blue disk with orange pollen.

2. Plants of the new Osteospermum have darker green leaves while plants of the cultivar ‘Sunny Zara’ have yellow-green-colored leaves.

3. Plants of the new Osteospermum are shorter and more compact than the plants of the cultivar ‘Sunny Zara’

4. Plants of the new Osteospermum have more flowers per plant than the plants of the cultivar ‘Sunny Zara’ as well as larger flowers.

Plants of the cultivar ‘Sunny Philip’ have not been observed under all possible environmental conditions. The phenotype may vary somewhat with variations in environment such as temperature, light intensity, day length, and fertility level without, however, any variance in genotype.

### BRIEF DESCRIPTION OF THE PHOTOGRAPHS

The accompanying color photographs illustrate the overall appearance and details of flower form color and structures of the new cultivar, showing the colors as true as it is reasonably possible to obtain in color reproductions of this type. Colors in the photographs may differ slightly from the color values cited in the detailed botanical description, which more accurately describe the actual colors of the new Osteospermum.

The first photograph shows a side perspective view of a typical flowering plant of ‘Sunny Philip’ as grown in an 11 cm pot.

The second photograph shows close-up, top view of the composite young and older flowers of ‘Sunny Philip’.

The third photograph shows a close-up of an individual flower of ‘Sunny Philip’.

The fourth photograph shows a close-up of the back side of an individual flower of ‘Sunny Philip’.

The fifth photograph shows the detail of front and back of a ‘Sunny Philip’ flower and young and older leaves.

The sixth photograph shows numerous ‘Sunny Philip’ plants as grown in the greenhouse.

### DETAILED BOTANICAL DESCRIPTION

In the following description, color references are made to The Royal Horticultural Society Colour Chart (RHS), 4^{th} edition. Plants were grown under greenhouse conditions. Plants used for this description were grown for about 20 weeks after cutting
Botanical classification: Osteospermum eclonis (DC) T. Norl.
Parentage:
   Female parent.—Osteospermum eclonis (DC) T. Norl. Seedling No. 3.122.98.
   Male parent.—Osteospermum eclonis (DC) T. Norl., Seedling No 9913.
   Propagation.—Type cutting terminal vegetative cuttings.
   Root description.—Fine, well branched.
Plant description:
   Form.—Perennial plant with upright plant habit. Osteospermum flowers in composite heads. Freely branching with lateral flowering branches forming at every node; dense and bushy.
   Crop time.—After rooting, about 18 weeks are required to produce finished flowering plants in 11 cm pots.
   Plant height (soil level to top of plant plane).—About 18 cm.
   Diameter.—25 cm.
   Vigor.—Moderately vigorous growth rate.
   Foliage description.—Leaves alternate, single, lobed, obovate to spatulate shape, brochidodromus venation. Length: 3-10 cm. Width: Up to 50 mm. Apex: obtuse. Base: attenuate. Margin: broadly dentate. Texture: smooth, glabrous, shiny. Scattered short, stiff hairs. Color: Young foliage, upper and lower surfaces: 137C and 137D, green. Mature foliage, upper surfaces 139A, lower surface 139B. Yellow-green flat, winged petioles.
Flower description:
   Flower arrangement and shape.—Tubular disk and petalled ray flowers in composite “pin-wheel” heads; with 15 parted involucre. 9-10 cm sturdy peduncles.
   Natural flowering season.—Continuous throughout the spring and summer. Season can be extended by vernalization and long day treatments.
   Flower longevity on the plant.—Longevity of individual flowers is highly dependent on temperature and light conditions 5 to 10 days. Flowers persistent, petals folding and withering slowly.
   Inflorescence diameter.—About 5 cm.
   Flowers.—Length: Ray petals: About 23 mm. Width: About 5 mm. Spatulate, acute ray petal lobes. Ray petal color: Upper surfaces, 155B, white to 85A violet at base, lower surfaces (some of which are seen from above) 85A to 86C violet. Disk florets tubular, 3 mm high, 86C Tips of corolla to translucent 155A at the base.
   Peduncle.—Strength: Moderately strong. Length: About 9-10 cm. Diameter: About 2 mm. Color: 144C yellow-green.
Reproductive organs:
   Anthers.—Linear, fused, stamen color 202A, black, pollen 16A yellow-orange.
   Style and stigma, color 155A,white and 201A black, respectively.
   Seed.—Length: About 1 mm. Diameter: About 0.3 mm.
   Weather tolerance.—Plants of the new Osteospermum have exhibited good tolerance to draught, rain.

1. A new and distinct cultivar of Osteospermum plant named ‘Sunny Philip’, as illustrated and described herein.
